(19) United States
(12) Patent Application Publication
(10) Pub. No.: US 2020/0138792 A1
(43) Pub. Date: May 7, 2020

(54) PHARMACEUTICAL COMBINATIONS OF ZONISAMIDE AND PRAMIPEXOLE, AND RELATED METHODS, FOR TREATING SYNUCLEINOPATHIES

(71) Applicant: CHASE THERAPEUTICS CORPORATION, Washington, DC (US)

(72) Inventors: Thomas N. CHASE, Washington, DC (US); Kathleen E. CLARENCE-SMITH, Washington, DC (US)

(73) Assignee: CHASE THERAPEUTICS CORPORATION, Washington, DC (US)

(21) Appl. No.: 16/612,805

(22) PCT Filed: May 23, 2018
(86) PCT No.: PCT/US2018/034050

§ 371 Date: Nov. 12, 2019

Related U.S. Application Data
(60) Provisional application No. 62/511,424, filed on May 26, 2017.

(51) Int. Cl. A61K 31/428; A61K 31/423; A61P 25/28; A61K 9/00; A61K 9/28; A61K 9/48

(52) U.S. Cl. CPC A61K 31/428; A61K 31/423; A61K 9/4891; A61K 9/0056; A61K 9/28; A61P 25/28

(57) ABSTRACT

The present invention relates to a combination of zonisamide with pramipexole or a pharmaceutically acceptable salt or solvate of pramipexole for treating a synucleinopathy.

### RELATED APPLICATIONS

This application claims the benefit of U.S. Provisional Patent Application Ser. No. 62/511,424, filed May 26, 2017, the disclosure of which is incorporated herein by reference in its entirety.

### FIELD OF THE INVENTION

The present invention pertains to the field of the treatment of synucleinopathies, i.e., of neurodegenerative disorders of the human central nervous system, and in particular of the treatment of neurotoxic processes due to the alpha-synuclein oligomerization and aggregation.

### OBJECTS OF THE INVENTION

The present invention concerns pharmaceutical combinations comprising zonisamide and pramipexole, or a pharmaceutically acceptable salt or solvate of pramipexole, and their use for the treatment of synucleinopathies, in particular of the CNS neurotoxic effects of alpha-synuclein in a human subject showing an abnormal plasma exosomal/total alpha-synuclein ratio in blood.

### Definitions

“CNS”: Central Nervous System.
“IR”: Immediate Release of the active ingredient from a composition.
“ER”: Extended Release of the active ingredient from a composition.
“TTS”: Transdermal Therapeutic System.
“AD”: Alzheimer's Disease.
“DLB”: Dementia with Lewy bodies.
“LBD”: Lewy Body Dementia.
“SNCA gene”: Synuclein-alpha or alpha-synuclein gene.
“Synucleinopathy”: A disease characterized by the abnormal accumulation, processing and spreading of alpha-synuclein (α-synuclein) in the brain. Namely, α-synuclein deposits in the central, peripheral, and autonomic nervous system. Synucleinopathies (also called α-synucleinopathies) are neurodegenerative diseases which include, but are not limited to Parkinsons' disease, Lewy body dementia (LBD) or dementia with Lewy bodies (DLB), Alzheimer's disease, the Lewy body variant of AD, multiple system atrophy, neurodegeneration with brain iron accumulation, and parkinsonian disorders associated with glucocerebrosidase (GBA) mutations.
“MSA”: Multiple System Atrophy.
“PD”: Parkinson's Disease.
“Zonisamide”: 1,2-benzisoxazole-3-methanesulfonamide.
“Effective daily dose of zonisamide”, as used herein, refers to a daily dose of zonisamide that is at least as high as an approved daily dose (200 mg to 600 mg per day) for the treatment of convulsions.
“Effective daily dose of pramipexole”, as used herein, refers to a daily pramipexole dose corresponding to at least a pramipexole dihydrochloride monohydrate approved daily dose (0.375 mg to 4.5 mg) for the treatment of PD.
The terms “comprise,” “comprises,” “comprising” “include,” “includes,” and “including” are interchangeable and not intended to be limiting. It is to be further understood that where descriptions of various embodiments use the term “comprising,” those skilled in the art would understand that the present disclosure also contemplates such embodiments alternatively described using the language “consisting essentially of” or “consisting of”.

### BACKGROUND OF THE INVENTION

Alpha-synuclein, a protein composed of 140 amino acids encoded by the SNCA gene, is abundantly expressed in the human brain and to a lesser extent in various other organs. In brain, alpha-synuclein (hereafter also referred to as simply “synuclein”) is mainly found in neuronal terminals, especially in the cortex, hippocampus, substantia nigra and cerebellum, where it contributes to the regulation of neurotransmitter release, and passes into the peripheral blood stream (Marques and Outeiro, 2012), in part, packaged within exosomal vesicles originating from the CNS (Shi et al, 2014).

Under normal circumstances, this soluble protein apparently forms a stably folded tetramer that resists aggregation. But, in certain pathological conditions, for unknown reasons, the alpha-synuclein misfolds, oligomerizes and aggregates (with the formation of fibrils or “fibrillization”), thus changing its conformation in an abnormal manner into one that is toxic and capable of gaining access to the peripheral (systemic) circulation.

The neurotoxic effects of the aberrant processing of alpha-synuclein into oligomerized and aggregated forms are now believed to contribute to the onset and subsequent progression of symptoms of various pathological conditions, notably PD, LBD, PD dementia, DLB, parkinsonian disorders associated with glucocerebrosidase (GBA) mutations, MSA, some forms of Alzheimer's disease, and several other disorders, herein collectively referred to as “synucleinopathies”. These are generally defined as a group of neurodegenerative disorders characterized in part by the intracellular accumulation of abnormal synuclein aggregates, some of which are toxic and contribute to the pathogenesis of PD, AD, and other neurodegenerative disorders (Kim et al. 2004).

The abnormal plasma exosomal/total alpha-synuclein ratio in the blood of a patient is a diagnostic hallmark of a synucleinopathy.

PD is a common neurodegenerative disorder of the human CNS, first described by James Parkinson in 1817. It has three major clinical signs: resting tremor, bradykinesia, and muscular rigidity. In addition, postural instability and various neurobehavioral disabilities may occur. In the US alone it is estimated that over 1 million individuals are afflicted by this inexorably progressive disorder. Moreover, PD prevalence continues to rise along with the general aging of the American population. Parkinsonian motor signs are now believed to largely reflect a progressive loss of dopaminergic neurons within the nigrostriatal system. The cause of this degenerative process remains incompletely understood, but now appears to involve the misprocessing of alpha-synuclein into abnormal neurotoxic species.

Lewy body dementia (LBD) is one of the most common types of progressive dementia. The central features of LBD include progressive cognitive decline, visual hallucinations, and parkinsonian motor symptoms, such as slowness of movement, difficulty walking, and muscular rigidity. Some may also suffer from depression. The symptoms of LBD are caused by the selective loss of nerve cells, presumably a result of synuclein misprocessing and associated with the build-up of Lewy bodies, spherical synuclein accumulations inside many of the degenerating neurons. Researchers do not know why alpha-synuclein accumulates into Lewy bodies or how synuclein species can cause the symptoms of LBD. The formation of Lewy bodies have been considered to be a marker for PD; however, Lewy bodies have also been observed in up to 60% of both sporadic and familial cases of Alzheimer's disease (AD) (Al-Mansoor et al. 2013). Accordingly, the aggregation of α-synuclein has been strongly implicated as a critical step in the development of neurodegenerative diseases (Al-Mansoor et al. 2013).

Sporadic PD or brainstem-predominant type LBD (PD dementia), and dementia with Lewy bodies (DLB) are the two most frequent α-synucleinopathies, and are progressive multisystem neurodegenerative disorders with widespread occurrence of α-synuclein deposits in the central, peripheral, and autonomic nervous system (Jellinger K A 2008a). Reportedly, there is considerable clinical and pathologic overlap between PD (with or without dementia) and DLB (or LBD), corresponding to Braak LB stages 5 and 6, both frequently associated with variable Alzheimer-type pathology (Jellinger K A 2008a). Dementia often does not correlate with progressed stages of LB pathology, but may also be related to concomitant Alzheimer lesions or mixed pathologies (Jellinger K A, 2008a).

Alzheimer disease (AD) has been reported to be featured by deposition of β-amyloid peptides, phosphorylated tau protein (3- and 4-repeat tau) and α-synuclein (aSyn) deposits (Jellinger K A, 2008b). Lewy body diseases (LBD), such as sporadic Parkinson disease (PD) and dementia with Lewy bodies (DLB), show aSyn-positive deposits in neurons, neurites, glia, and presynaptic terminals, while frontotemporal dementias present tau-positive and tau-negative, ubiquitin- and TDP-43-positive neuronal and glial inclusions (Jellinger K A, 2008b). Molecular interactions between major proteins, which may occur within the same brain in various distribution patterns, are associated with various phenotypes and mixed pathologies, e.g. AD with aSyn pathology in the brainstem and amygdala, PD and DLB with AD lesions, and frontotemporal dementia with a mixture of various deposits, while others are featured by one principal pathology without other lesions (e.g. tangle-predominant type of dementia, pure PD, brainstem-predominant LBD) (Jellinger K A, 2008b).

MSA with orthostatic hypotension is the current term for a neurological disorder that was once called Shy-Drager syndrome. A progressive disorder of the central and autonomic nervous systems, it is characterized by orthostatic hypotension (an excessive drop in blood pressure when standing up), which causes dizziness and fainting. MSA can occur without orthostatic hypotension, but instead have urinary tract involvement (urgency/incontinence). Neurologists classify the disorder into 3 types: the parkinsonian-type includes symptoms of PD such as slow movement, stiff muscles, and tremor; the cerebellar-type, which causes problems with coordination and speech; and the combined-type, which includes symptoms of both parkinsonism and cerebellar dysfunction. Problems with urinary incontinence, constipation, and sexual impotence may happen early in the course of the disease. Other symptoms include generalized weakness, double vision or other vision disturbances, difficulty breathing and swallowing, sleep disturbances, and decreased sweating. Because the disease resembles others, a correct diagnosis may take years.

Mutations in the glucocerebrosidase gene (GBA) can result in the autosomal recessive disorder Gaucher disease. Different lines of evidence suggest that mutant GBA may be a risk factor for Parkinson's disease. GBA mutations are now thought to be the single largest risk factor for development of idiopathic PD. Clinically, on imaging and pharmacologically, GBA PD is almost identical to idiopathic PD (O'Regan et al, 2017). The molecular mechanisms which lead to this increased PD risk in GBA mutation carriers are not fully elucidated, but have been shown to be associated with accumulation of synuclein (Soria et al, 2017).

Several other disorders have also, albeit less frequently, been considered synucleinopathies. These include Hallevorden-Spatz syndrome, neuronal axonal dystrophy, and some cases of traumatic brain injury. In the case of Hallevorden-Spatz, symptoms include parkinsonism, dystonia, dysphagia/dysarthria, rigidity or stiffness of the limbs, spasticity, and dementia.

Many now believe that processes leading to synuclein oligomerization and aggregation may be central to the neural injury and destruction occurring in these synucleinopathic disorders.

The mechanism of alpha-synuclein aggregation in the synucleinopathies remains uncertain. Current evidence suggests the conversion of an alpha helical structure into a beta pleated conformation and subsequent oligomerization might be the pathogenic antecedents to the aggregation and fibrillization of synuclein. These characteristics are similar to the aberrant processing of prion proteins that also can become highly neurotoxic. Phosphorylation of alpha-synuclein at the serine-129 residue has been implicated as a contributory factor (Chen et al. 2016). According to this author, a prion form of alpha-synuclein could be a causal agent, especially for multiple system atrophy. Prions are small proteins that also can misfold, oligomerize, aggregate and propagate to other cells. The result in brain is a profound and spreading neurotoxic process.

Accordingly, inhibiting the misfolding, oligomerization and aggregation of synuclein may be beneficial in favorably modifying synucleinopathic disorders, such as in slowing or even arresting the progression of synucleinopathic disorders.

As mentioned above, alpha-synuclein readily passes into extracellular spaces and has been identified in cerebrospinal fluid, blood, and saliva (Marques and Outeiro, 2012). The mechanisms of alpha-synuclein secretion have yet to be fully elucidated, although recent studies have demonstrated that at least a fraction of alpha-synuclein is secreted in association with exosomes, 40 nm to 100 nm vesicles of endocytic origin (reviewed in Shi et al. 2014). Plasma exosomal alpha-synuclein as well as certain of the oligomerized and aggregated products of its misprocessing into toxic species have been reported to correlate with disease severity (Shi et al. 2014). Accordingly, peripheral plasma exosomal alpha-synuclein and related species can help monitor central disease progression as well as the effects of drugs, such as pramipexole, on the rate and degree of disease modification (Luo et al. 2016).

Similarly, exosomal alpha-synuclein levels correlated with severity of impairment in cross-sectional samples from patients with LBD (Stuendl et al. 2016).

Based on the above, drugs that correct abnormalities in plasma exosomal alpha-synuclein species will delay onset and slow progression of the neuronal destruction associated with the synucleinopathies.

Various compositions for the treatment of PD and related disorders that target the synuclein aggregation pathway have been proposed. The discovery process primarily involves cellular and animal models of prion and synuclein induced neurodegeneration (Prusiner S B 2015; Visanji N P et al 2016). Unfortunately, none of these models has been validated and all are deemed relatively uncertain predictors of effects in humans. Nevertheless, these models continue to be widely used in the absence of more reliable discovery techniques.

Pharmaceutical agents currently proposed for the treatment of synucleinopathies include, such small molecules as zonisamide and pramipexole. Zonisamide (1,2-benzisoxazole-3-methanesulfonamide) is a sulfonamide anticonvulsant approved for use in the adjunctive therapy of adults with partial-onset seizures; including infantile spasm, mixed seizure types of Lennox-Gastaut syndrome, myoclonic, and generalized tonic clonic seizure.

This drug is commercially available (Zonegran®) and is supplied for oral administration as capsules containing 25 mg or 100 mg zonisamide. In the treatment of epilepsy, oral zonisamide is generally used in daily doses of 200 mg to 600 mg per day, divided in 2 daily doses, and adjusted to maintain serum levels of 15 to 40 micrograms/milliliter.

The drug, unrelated to other anticonvulsants, is believed to act, at least in part, by blocking voltage dependent sodium and T-type calcium channels. It is also a weak carbonic anhydrase inhibitor and a modulator of brain GABAergic and glutamatergic neurotransmission.

Zonisamide has been reported to exhibit protective activity in various neurotoxin-based cellular and animal models of PD. In 1-methyl-4-phenyl-1,2,3,6-tetrahydropyridine (MPTP)-treated mice, zonisamide administration (20 mg/kg) reduced the loss of nigral TH-positive neurons while attenuating the associated striatal dopamine depletion (Yokoyama H et al. 2010). In mice that received 6-hydroxydopamine (6-OHDA) to induce hemiparkinsonism, the injection of zonisamide (30 mg/kg) prevented the loss of nigral dopamine neurons. In more direct relation to synucleinopathic neurodegeneration, zonisamide inhibits the in vitro oligomerization and aggregation of alpha-synuclein, a key event in pathogenesis of PD and related disorders (Effects of antiparkinsonian agents on β-amyloid and α-synuclein oligomer formation in vitro. Ono K et al. 2013) and exerts protective effects against A53T α-synuclein-induced neurodegeneration in a manner that may be independent of synuclein aggregation in an in vivo rat model (Arawaka S et al. 2014). In the latter study, orally administered zonisamide (40 mg/kg/day) significantly delayed the pace of degeneration four weeks after injection of the viral vector for the A53T α-synuclein gene as compared with the control group. This effect lasted at least eight weeks after transgene injection, but appeared to have no impact on the survival of nigrostriatal dopamine neurons. The chronic administration of zonisamide to Engrailed mutant mice, another genetic model of PD, improved the survival of nigrostriatal dopaminergic neurons as well as their striatal dopaminergic terminals and motor function as compared with saline treatment (Sano H et al 2015). The mechanism of these protective effects remains uncertain, although brain-derived neurotrophic factor content reportedly increased in the striatum and ventral midbrain of the zonisamide-treated mice compared to saline-treated controls.

Zonisamide, given at relatively low doses (25 mg-50 mg) either alone or with levodopa, has been observed to improve motor symptoms in patients with PD (Grover N D et al. 2013). However, there are no clinical reports documenting that zonisamide exerts disease modifying effects in humans with a neurodegenerative disease such as PD, or modifies synuclein species in blood exosomes from patients with PD type disorders.

Another pharmaceutical agent currently proposed for consideration is pramipexole and its analogues, alone or in combination with various drugs.

Pramipexole is a synthetic aminothiazole derivative described in U.S. Pat. No. 4,886,812, the content of which is incorporated herein in its entirety by reference. It is a dopamine autoreceptor agonist (Schneider C S and Mierau J, 1987) of the non-ergoline class that is approved, as pramipexole dihydrochloride monohydrate, for the treatment of PD (see Mirapex® Package Insert; Revised July 2016), in doses ranging from 0.375 mg/day to 4.5 mg/day, given in 3 equally divided doses (Mirapex® Prescribing Information, July 2016). Pramipexole is supplied in tablets containing 0.125 mg, 0.25 mg, 0.5 mg, 1 mg and 1.5 mg of pramipexole dihydrochloride monohydrate, in an IR-formulation, and in tablets containing 4.5 mg of pramipexole dihydrochloride monohydrate, in an ER-formulation.

Thus, the compound that is actually present in the pharmaceutical formulation and administered to animals and patients is pramipexole dihydrochloride monohydrate (USAN: pramipexole dihydrochloride), even though it is often simply called pramipexole in the scientific literature.

Although widely used for the relief of parkinsonian symptoms, its potential as a disease modifying agent has made it the object of considerable investigative attention.

Pramipexole reportedly diminishes synuclein oligomer formation in vitro (Ono et al. 2013). Related studies suggest that pramipexole inhibits the toxic effects of rotenone on dopaminergic neurons in a mouse PD model while reducing immunoreactivity for alpha-synuclein; additionally, pramipexole decreases the in vitro oligomerization of human wild-type alpha-synuclein by hydrogen peroxide plus cytochrome c (Inden et al. 2009). Pramipexole has also been observed to inhibit the aggregation of alpha-synuclein in human neuroblastoma SH-SY5Y cells (Kakimura et al. 2009). Importantly, the relative expression of α-synuclein in serum exosomes has been found to decline during pramipexole treatment of PD-type patients (Luo et al. 2016).

Unfortunately, limitations associated with the administration of pramipexole to synucleinopathic patients complicate its use at the potentially higher neuroprotective doses that could be extrapolated from some animal models. First, mechanisms to explain its putatively beneficial effects on synuclein-related neurotoxicity continue to elude full understanding. Second, effect sizes in animal model studies tend to be small and occur only at relatively high drug doses. Both situations were also observed in the above mentioned report of pramipexole-induced changes in exosomal synuclein in PD patients, which were associated with the administration of the highest—4.5 mg/day—recommended/approved dose of pramipexole (Mirapex Package Insert; Revised July 2016).

In the report by Luo et al. (2016), although treatment of Parkinson patients with pramipexole at therapeutic doses significantly lowered the relative expression of alpha-synuclein (compared with pre-treatment values), the magnitude of the effect was small. The use of higher doses of pramipexole was precluded by side effects such as vomiting and severe nausea. For example, Corrigan et al (2000) report that doses of 5 mg/day of pramipexole, hardly higher than the maximum recommended dose of 4.5 mg/day (Pramipexole FDA-approved package Insert) caused nausea in 76% of patients and vomiting in 39% of patients. Furthermore, 36% of patients were not able to complete the study, presumably because of intolerable GI adverse events.

The US 2003/0032661 patent document, the content of which is incorporated herein in its entirety by reference, discloses the use of pramipexole therapeutically effective doses for the prevention and/or treatment of generalized seizures (absences, also atypical absences, myoclonic, clonic, tonic, and tonic-clonic seizures), focal (simple and complex focal) and secondary generalized seizures; and affirms that pramipexole can be used in therapeutically effective doses as an anticonvulsant for treating said cerebral seizures. According to this document, pramipexole may be used in doses of about 0.05 mg to 7.5 mg, preferably 0.1 mg to 5 mg per day, at maximum doses of about 5 mg to 7.5 mg of pramipexole per day. In addition, this document affirms that pramipexole may be used to treat the abovementioned conditions in conjunction, for example, with one or more, preferably one of the following substances: carbamazepine, oxcarbamazepine, valproic acid, diphenylhydantoin, ethosuximide, mesuximide, phenobarbital, primidone, benzodiazepines (preferably diazepam, clonazepam or clobazam), corticotrophin, corticoids, bromides (such as potassium bromide), sultiam, acetazolamide, felbamate, gabapentin, lamotrigine, topiramate, vigabatrin, levetiracetam, and zonisamide. This document, however, does not give any information about how said pramipexole and, a fortiori, any combination thereof with other drugs would have been used for treating said seizures, and limits the description of the pramipexole unit forms to a maximum 1 mg strength.

Thus, zonisamide has not been used, nor suggested for use, in combination with pramipexole for treating PD or any other synucleinopathy.

In conclusion, the state of the art shows that (a) the efficacy of pramipexole in the treatment of PD is insufficient, (b) the fact that pramipexole possesses a disease modifying ability in patients with a synuclein related disorder has not been clinically proven, and (c), as set forth above, said efficacy is limited by the adverse effects of this drug.

Thus, more than twenty years after the approval of Mirapex®, eighteen years after the approval of Zonergan®, and fifteen years after the publication of US 2003/0032661, the problem of providing safe, chronic, effective treatment of a patient suffering from a synucleinopathy with pramipexole remains unresolved.

### SUMMARY OF THE INVENTION

The present invention increases the therapeutic window for pramipexole to safely enhance its efficacy to a degree that delays onset and/or slows symptom progression in those suffering from a synucleinopathy. One approach to this end is to administer a synergistic drug combination comprising pramipexole and an anticonvulsant drug that reduces the minimum effective dose for pramipexole and/or increases pramipexole's effect magnitude without diminishing its minimum toxic dose. An example of such a synergistic drug is the anticonvulsant drug zonisamide.

The present inventors found that zonisamide acts by augmenting the synucleinopathy-modifying potential of pramipexole in humans, thus allowing at least a slowing of the disease progression at doses that are both safe and tolerable.

The combination of zonisamide with pramipexole or a pharmaceutically acceptable salt or solvate of pramipexole, acts to normalize levels of synuclein species in the plasma of patients suffering from a synucleinopathy, in particular by diminishing the concentration of abnormal synuclein species (congeners) in the patient's plasma and in the exosomal vesicles found therein, to a significant degree at doses that are safe and tolerable thus evidencing that said patients will enjoy neuroprotective benefit.

This observation is unexpected since neither drug has been found clinically to possess any disease modifying ability in patients with a synuclein related disorder. Moreover, nowhere has it even been suggested that the combination of these drugs might confer such significant benefit to such individuals since the antiparkinson drug and the anticonvulsant drug primarily act by different mechanisms.

The present invention is based on the discovery that
zonisamide potentiates (augments) the ability of pramipexole to alter blood exosomal synuclein species in ways indicating the activation of a central neuroprotective mechanism, i.e. reducing oligomerization of synuclein;
these changes occur at safe and tolerable doses of both drugs; and
these changes are indicative of CNS changes that confer clinical improvement in a way and to degree that will provide practical and significant disease modifying benefit to sufferers.

The findings are surprising since the two drugs are thought to act on different targets, by different mechanisms, to produce different clinical effects, and thus would hardly be expected to have effects in the manner disclosed herein.

Thus, the present invention provides zonisamide, for use for the treatment of synucleinopathies in patients in need of said treatment, in combination with pramipexole or a pharmaceutically acceptable salt or solvate thereof; and the use of zonisamide for the preparation of a medicament for the treatment of a synucleinopathy in combination with pramipexole or a pharmaceutically acceptable salt or solvate thereof.

The invention further provides a method for treating a patient suffering from a synucleinopathy, which comprises treating said patient with zonisamide in combination with pramipexole or a pharmaceutically acceptable salt or solvate thereof.

Preferably, the method for treating a patient suffering from a synucleinopathy according to the present invention comprises treating said patient with an effective daily dose of zonisamide in combination with an effective daily dose of pramipexole dihydrochloride monohydrate.

Said effective daily dose of pramipexole, including low doses used in the titration period, is from 0.375 mg to 20 mg, normally from 0.375 mg to 6 mg.

According to an embodiment, in the above use and method, zonisamide and pramipexole or a pharmaceutically acceptable salt or solvate of pramipexole are each formulated in a pharmaceutical composition in admixture with a pharmaceutical carrier and concurrently or sequentially administered to the patient in need of said treatment.

According to another embodiment, zonisamide and pramipexole or a pharmaceutically acceptable salt or solvate of pramipexole are mixed together and formulated in a pharmaceutical composition (fixed-dose combination) in admixture with a pharmaceutical carrier. This composition is destined to be administered to a patient suffering from a synucleinopathy.

The above pharmaceutical compositions are normally formulated in unit forms, each unit form comprising
zonisamide, in admixture with a pharmaceutical carrier or vehicle; or
pramipexole or a pharmaceutically acceptable salt or solvate thereof, in admixture with a pharmaceutical carrier or vehicle; or
zonisamide and pramipexole or a pharmaceutically acceptable salt or solvate of pramipexole, mixed together and in admixture with a pharmaceutical carrier or vehicle in the same unit form.

Zonisamide is present in said pharmaceutical compositions in an amount/unit form of from 25 mg to 600 mg and pramipexole or pharmaceutically acceptable salt or solvate thereof is present in said pharmaceutical compositions in an amount per unit form that is equivalent to from 0.125 mg to 6 mg. of pramipexole dihydrochloride monohydrate.

Advantageously, said pramipexole is present in said compositions in an amount per unit form of from 0.125 mg to 20 mg.

According to an advantageous embodiment, in the method (or use) for the treatment of a synucleinopathy, zonisamide is formulated in a pharmaceutical composition, in the aforementioned amount per unit form, in admixture with a pharmaceutical carrier or vehicle. This composition is administered to a patient in need of said treatment at a daily dose of from 25 mg to 600 mg of zonisamide, in combination with pramipexole or pharmaceutically acceptable salt or solvate thereof. Said pramipexole (or pramipexole pharmaceutically acceptable salt or solvate) is also formulated in a pharmaceutical composition, in the aforementioned amount per unit form, in admixture with a pharmaceutical carrier or vehicle and is administered to said patient in need of said treatment at a daily dose that is equivalent to from 1.5 mg to 6 mg of pramipexole dihydrochloride monohydrate. Daily doses equivalent to from 1.5 mg to 20 mg, from 1.5 mg to 15 mg, from 1.5 mg to 10 mg, and from 1.5 mg to 7.5 mg of pramipexole dihydrochloride monohydrate are also provided.

In practice, in the above combination, use and method, zonisamide is present in said composition in an amount per unit form of from 25 mg to 600 mg, in particular in an amount of from 25 mg to 200 mg in IR-unit form and from 200 mg to 600 mg in ER-form.

Pramipexole or a pharmaceutically acceptable salts and solvates thereof is present in a dose per unit form equivalent to from 0.125 mg to 20 mg, from 0.125 mg to 15 mg, from 0.125 mg to 10 mg, from 0.125 mg to 7.5 mg, from 0.125 mg to 6 mg, or from 0.125 ng to 3 mg of pramipexole dihydrochloride monohydrate. Preferably said pramipexole salt or solvate is pramipexole dihydrochloride monohydrate.

In an IR-unit form, pramipexole is present in an amount equivalent to a range selected from the group consisting of from 0.125 mg to 10 mg, from 0.125 mg to 7.5 mg, from 0.125 mg to 5 mg, from 0.125 mg to 3.75 mg, and from 0.125 mg to 3 mg of pramipexole dihydrochloride monohydrate.

In an ER-unit form, pramipexole is present in an amount equivalent to a range selected from the group consisting of 0.375 mg to 20 mg, from 0.375 mg to 15 mg, from 0.375 mg to 10 mg, from 0.375 mg to 7.5 mg, and from 0.375 mg to 6 mg of pramipexole dihydrochloride monohydrate.

In an advantageous method (or use), said zonisamide and pramipexole combination is administered to said patient suffering from a synucleinopathy in a fixed-dose combination wherein said zonisamide and said pramipexole or pharmaceutically acceptable salt or solvate of pramipexole, are mixed together and with a pharmaceutical carrier or vehicle.

Preferably, the pramipexole or pharmaceutically acceptable salt or solvate thereof is pramipexole dihydrochloride monohydrate.

### DETAILED DESCRIPTION

The present invention is based on the discovery that the pharmacological agent zonisamide synergistically and substantially improves the ability to provide safe and tolerable doses of pramipexole to reduce toxic synuclein oligomers in plasma exosomes of patients with PD and related synucleinopathic disorders, and thus benefit patients with such fatal disorders to a previously unrealized degree.

In particular, the present invention provides a synergistic pharmaceutical combination comprising, as Components,

(a) zonisamide; and
(b) pramipexole or a pharmaceutically acceptable salt or solvate thereof, for the treatment of a synucleinopathy.

More particularly, the present invention provides
zonisamide, for use in the treatment of a synucleinopathy in combination with an effective daily dose of pramipexole or of a pharmaceutically acceptable salt or solvate thereof;
the use of zonisamide for the preparation of a medicament for the treatment of synucleinopathies in combination with pramipexole or of a pharmaceutically acceptable salt or solvate thereof; and
a method for the safe treatment of a synucleinopathy in a patient in need of said treatment, which comprises administering to said patient an effective daily dose of zonisamide in combination with an effective daily dose of pramipexole or of a pharmaceutically acceptable salt or solvate thereof.

Herein below, the expressions “salt or solvate thereof”, “salts or solvates thereof” and “salts and solvates thereof”, in reference to pramipexole, indicate that the salt of said pramipexole may be solvated with a solvent, normally water.

Unless otherwise specified, “pramipexole” generally stands for (S)-6-propylamino-4,5,6,7-tetrahydro-1,3-benzothiazole-2-amine, as free base (pramipexole) or as a pharmaceutically acceptable salts and solvates thereof, including pramipexole dihydrochloride monohydrate, their doses per unit form or their daily doses being expressed as equivalents of pramipexole dihydrochloride monohydrate.

Pharmaceutically acceptable salts or solvates of pramipexole are also included in the present invention. Illustrative examples of these salts include acid addition salts with mineral acids such as hydrochloric acid, hydrobromic acid, hydriodic acid, sulfuric acid, nitric acid, phosphoric acid and the like or with organic acids such as formic acid, acetic acid, propionic acid, oxalic acid, malonic acid, succinic acid, fumaric acid, maleic acid, lactic acid, malic acid, tartaric acid, citric acid, carbonic acid, methanesulfonic acid, ethanesulfonic acid, aspartic acid, glutamic acid and the like. The solvation agent is generally water.

### First Aspect of the Invention

According to a first aspect, the present invention provides zonisamide, for use in the treatment of a synucleinopathy, in a synergistic combination with an effective daily dose of pramipexole.

A first embodiment of this first aspect provides a pharmaceutical combination comprising zonisamide, as Component (a), in a pharmaceutical composition comprising, as an active ingredient, said zonisamide, in admixture with a pharmaceutical carrier or vehicle, to be administered in combination with pramipexole or a pharmaceutically acceptable salt or solvate thereof, as Component (b), also in a pharmaceutical composition in admixture with a pharmaceutical carrier or vehicle.

A second embodiment provides a pharmaceutical fixed-dose combination consisting of a pharmaceutical composition comprising zonisamide, as Component (a), and pramipexole or a pharmaceutically acceptable salt or solvate thereof, as Component (b), in admixture with a pharmaceutical carrier or vehicle.

According to this first aspect of the present invention, said zonisamide, Component (a), is present in said combination in an amount per unit form that is at least as high as a dose per unit form approved the treatment of convulsions; and said pramipexole or pharmaceutically acceptable salt or solvate thereof, Component (b), is present in said combination in an amount per unit form (in pramipexole dihydrochloride monohydrate) that is at least as high as a dose per unit form approved for the treatment of PD.

Normally, said zonisamide is present in said composition in an amount per unit form of from 25 mg to 600 mg, in particular in an amount of from 25 mg to 200 mg in IR-unit form and from 200 mg to 600 mg in ER-form.

In the pharmaceutical composition to be administered in combination with the above zonisamide pharmaceutical composition, pramipexole or pharmaceutically acceptable salts and solvates thereof is present in a dose per IR or ER unit form equivalent to from 0.125 mg to 20 mg, from 0.125 mg to 15 mg, from 0.125 mg to 10 mg, from 0.125 mg to 7.5 mg, and from 0.125 mg to 6 mg of pramipexole dihydrochloride monohydrate. Preferably said pramipexole salt or solvate is pramipexole dihydrochloride monohydrate.

In general, said pramipexole dose per IR-unit form is equivalent to a range selected from the group consisting of from 0.125 mg to 10 mg, from 0.125 mg to 7.5 mg, from 0.125 mg to 5 mg, from 0.125 mg to 3.75 mg, and from 0.125 mg to 3 mg of pramipexole dihydrochloride monohydrate; and said pramipexole dose per ER-unit form is equivalent to a range selected from the group consisting of 0.375 mg to 20 mg, from 0.375 mg to 15 mg, from 0.375 mg to 10 mg, from 0.375 mg to 7.5 mg, and from 0.375 mg to 6 mg of pramipexole dihydrochloride monohydrate

In particular, in the combination according to this first aspect of the invention,
said zonisamide Component (a) is in a pharmaceutical composition comprising, as an active ingredient, said zonisamide, in an amount per unit form of from 25 mg to 600 mg, in admixture with a pharmaceutical carrier or vehicle; and
said pramipexole or a pharmaceutically acceptable salt or solvate thereof Component (b) is in a pharmaceutical composition comprising, as an active ingredient, said pramipexole or pharmaceutically acceptable salt or solvate thereof, in an amount per unit form equivalent to from 0.125 mg to 6 mg of pramipexole dihydrochloride monohydrate, in admixture with a pharmaceutical carrier or vehicle.

More particularly, in said combination of pharmaceutical compositions, zonisamide is present in an amount per unit form of from 25 mg to 200 mg in IR-form or in an amount per unit dose of from 200 mg to 600 mg in ER-form. Pramipexole is present, in IR-form, in an amount per unit form equivalent to from 0.125 mg to 1.5 mg of pramipexole dihydrochloride monohydrate, or, in ER-form, in an amount per unit dose equivalent to from 0.375 mg to 6 mg or from 1.5 mg to 6 mg of pramipexole dihydrochloride monohydrate.

### Second Aspect of the Invention

A second aspect of the present invention provides the use of zonisamide for the preparation of a medicament for the treatment of a synucleinopathy in combination with pramipexole or pharmaceutically acceptable salt or solvate thereof.

Said use of zonisamide normally is for the preparation of a medicament for the treatment of a synucleinopathy in combination with pramipexole or pharmaceutically acceptable salt or solvate thereof, said medicament normally consisting of a pharmaceutical composition in dosage unit form comprising, as an active ingredient, said zonisamide in admixture with a pharmaceutical carrier or vehicle.

As mentioned above, said medicament is destined to the treatment of a patient suffering from a synucleopathy such as PD, PD dementia, Lewy body dementia, Dementia with Lewy bodies, Alzheimer's disease, the Lewy body variant of Alzheimer's disease, multiple system atrophy, neurodegeneration with brain iron accumulation, and parkinsonian disorders associated with glucocerebrosidase (GBA) mutations.

In particular, according to this second aspect, the invention provides the use of zonisamide for the preparation of a medicament, as a pharmaceutical composition in dosage unit form comprising said zonisamide Component (a), in an amount per unit form at least as high as a dose per unit form approved for the treatment of convulsions, in admixture with a pharmaceutical carrier, for the treatment of a synucleinopathy, in combination with pramipexole or a pharmaceutically acceptable salt or solvate thereof Component (b) in doses, in pramipexole dihydrochloride monohydrate, at least as high as a dose approved for the relief of the motor symptoms of PD.

More particularly, according to this second aspect, the invention provides the use of zonisamide Component (a) for the preparation of a medicament consisting of a pharmaceutical composition in dosage unit form comprising, as an active ingredient, said zonisamide, in an amount per unit form of from 25 mg to 600 mg, in admixture with a pharmaceutical carrier or vehicle, for the treatment of a synucleinopathy in combination with pramipexole or a pharmaceutically acceptable salt thereof Component (b), also in a pharmaceutical composition comprising said with pramipexole or a pharmaceutically acceptable salt thereof in an amount of from 0.125 mg to 20 mg.

In particular, according to this second aspect, pramipexole or pharmaceutically acceptable salt thereof is in pharmaceutical composition, in an amount per unit form equivalent to 0.125 mg to 6 mg in admixture with a pharmaceutical carrier or vehicle.

Said pramipexole or pharmaceutically acceptable salt thereof in said composition is administered to said patient at a daily dose equivalent to from 0.375 mg to 20 mg, or from 0.375 mg to 6 mg. normally from 1.5 mg to 6 mg of pramipexole dihydrochloride monohydrate.

For this use, zonisamide is formulated in a pharmaceutical composition comprising from 25 mg to 600 mg of zonisamide, in admixture with a pharmaceutical carrier or vehicle and is administered to said patient at a daily dose of from 25 mg to 600 mg.

According to an embodiment, said medicament is a pharmaceutical composition in dosage unit form comprising, as an active ingredient, said zonisamide, in a fixed dose combination with, as another active ingredient, said pramipexole or pharmaceutically acceptable salt or solvate thereof, in admixture with a pharmaceutical carrier or vehicle.

Thus, this second aspect of the invention also provides the use of zonisamide for the preparation of a medicament consisting of pharmaceutical composition in dosage unit form comprising, as an active ingredient, said zonisamide Component (a), in an amount per unit form of from 25 mg to 600 mg, in a fixed-dose combination with, as another active ingredient, pramipexole or a pharmaceutically acceptable salt thereof Component (b), in an amount per unit form equivalent to from 0.125 mg to 20 mg of pramipexole dihydrochloride monohydrate, in admixture with a pharmaceutical carrier or vehicle, for the treatment of synucleinopathies.

In particular, according to this embodiment of this second aspect, said pharmaceutical composition comprises said zonisamide, in an amount of from 25 mg to 200 mg and pramipexole or pharmaceutically acceptable salt or solvate thereof, in an amount equivalent to from 0.125 mg to 6 mg of pramipexole dihydrochloride monohydrate, in admixture with a pharmaceutical carrier or vehicle.

### Third Aspect of the Invention

According to a third aspect, the present invention provides a method for treating a patient suffering from a synucleinopathy, which comprises administering to a patient in need of said treatment an effective yet tolerable daily dose of zonisamide in combination with an effective daily dose of pramipexole or a pharmaceutically acceptable salt thereof.

In the method (or use) for the treatment of a synucleinopathy, zonisamide is formulated in a pharmaceutical composition in dosage unit form comprising from 25 mg to 600 mg of zonisamide, in admixture with a pharmaceutical carrier or vehicle. This composition is administered to a patient in need of said treatment at a daily dose of from 25 mg to 600 mg, in combination with pramipexole or pharmaceutically acceptable salt or solvate thereof.

Said pramipexole is also formulated in a pharmaceutical composition in admixture with a pharmaceutical carrier or vehicle and is administered to said patient in need of said treatment at a daily dose that is equivalent to from 0.375 mg to 20 mg, normally from 1.5 mg to 6 mg of pramipexole dihydrochloride monohydrate.

In the above combination, use and method, the dose of zonisamide, per IR-unit form, will be in an amount of from 25 mg to 200 mg, and, in an ER-formulation, including slow-release compositions and transdermal therapeutic systems such as transdermal patches in an amount of from 200 mg to 600 mg.

In the above combination, use and method, pramipexole or a pharmaceutically acceptable salt or solvate thereof is formulated in a pharmaceutical composition in dosage unit form comprising said pramipexole or a pharmaceutically acceptable salt or solvate thereof in an amount per unit form equivalent to from 0.125 mg to 20 mg of pramipexole dihydrochloride monohydrate, in admixture with a pharmaceutical carrier or vehicle.

The dose of pramipexole or pharmaceutically acceptable salt or solvate thereof, per IR-unit form, will range from 0.125 mg to 10 mg, normally from 0.125 mg to 3 mg (in pramipexole dihydrochloride monohydrate).

The dose per unit form of pramipexole or a pharmaceutically acceptable salt or solvate thereof in an ER formulation, including slow-release compositions and transdermal therapeutic systems such as transdermal patches, will be equivalent to a range of from 0.375 mg to 20 mg, preferably from 1.5 mg to 20 mg (depending on the tolerability in combination with zonisamide, at the above dose per unit form), or, in some cases, from 0.375 mg to 6 mg, preferably from 1.5 mg to 6 mg.

### The Formulations

As illustrated above, the combination of the invention comprises
(a) zonisamide; and
(b) pramipexole or a pharmaceutically acceptable salt or solvate thereof, each in a pharmaceutical composition in admixture with a pharmaceutical carrier or vehicle; or
(a/b) zonisamide and pramipexole or a pharmaceutically acceptable salt or solvate of pramipexole, mixed together and in admixture with a pharmaceutical carrier or vehicle, as a fixed dose combination.

In the pharmaceutical compositions of the present invention for oral, subcutaneous, intravenous, transdermal or topical administration, the active ingredients are preferably administered in the form of dosage units, in admixture with the classic pharmaceutical carriers or vehicles.

In particular, in the above combination, use and method, zonisamide is present in said composition in an amount per unit form of from 25 mg to 600 mg, in particular in an amount of from 25 mg to 200 mg in IR-unit form and from 200 mg to 600 mg in ER-form.

Pramipexole or a pharmaceutically acceptable salts and solvates thereof is present in a dose per unit form equivalent to from 0.125 mg to 20 mg, from 0.125 mg to 15 mg, from 0.125 mg to 10 mg, from 0.125 mg to 7.5 mg, from 0.125 mg to 6 mg, or from 0.125 ng to 3 mg of pramipexole dihydrochloride monohydrate. Preferably said pramipexole salt or solvate is pramipexole dihydrochloride monohydrate.

In an IR-unit form, pramipexole is present in an amount equivalent to a range selected from the group consisting of from 0.125 mg to 10 mg, from 0.125 mg to 7.5 mg, from 0.125 mg to 5 mg, from 0.125 mg to 3.75 mg, and from 0.125 mg to 3 mg of pramipexole dihydrochloride monohydrate.

In an ER-unit form, pramipexole is present in an amount equivalent to a range selected from the group consisting of 0.375 mg to 20 mg, from 0.375 mg to 15 mg, from 0.375 mg to 10 mg, from 0.375 mg to 7.5 mg, and from 0.375 mg to 6 mg of pramipexole dihydrochloride monohydrate.

The treatment of the synucleinopathy is made according to the protocols established for the treatment of a patient with zonisamide or pramipexole in their own indications.

Thus, for example, when zonisamide is administered in its anticonvulsant indication, serum level monitoring is recommended for all patients to ensure appropriate dosing because
patient response correlates with zonisamide serum level;
serum level does not correlate with dose because of concentration-dependent erythrocyte binding;
elimination is affected by co-administration of drugs that affect NAT 1, CyP3A4, and UDPG; and
renal function affects elimination.

Steady-state zonisamide concentration in a trough specimen drawn just before next dose correlates with patient response, but not with dose. Optimal anticonvulsant responses to zonisamide occur when trough zonisamide concentrations are in the range of 10 mcg/mL to 40 mcg/mL. Peak serum concentration for zonisamide occurs 2 to 6 hours after dose, and time to peak is affected by food intake.

A titration is normally made when beginning the treatment with pramipexole of a patient suffering from PD, for the relief of the motor symptoms caused by PD, by administering low doses of pramipexole (0.375 mg/day) and augmenting said dose to reach the maximum tolerated dose or the maximum recommended dose (4.5 mg/day) for that indication.

The dosage, i.e. the amount of active ingredient in a single dose to be administered to a patient suffering from a synucleinopathy, can vary widely depending on the age, weight, and the health condition of the patient, as also described herein above. This dosage includes the administration of a single dose from 25 mg to 600 mg of zonisamide, and of a single dose of pramipexole or a pharmaceutically acceptable salt or solvate thereof that is equivalent to from 0.125 mg to 20 mg or from 0.125 mg to 6 mg of pramipexole dihydrochloride monohydrate, according to the age of the patient, from one to three times a day by intravenous, subcutaneous, oral, or transcutaneous administration, according to the strength of the doses of each of the active ingredients.

The pharmaceutical compositions of the present invention are formulated with the classic excipients suitable for different ways of administration. Particularly advantageous are the formulations in the form of tablets, multi-score tablets, coated tables, orally disintegrating tablets, extended release tablets, hard or soft capsules, extended-release capsules, patches for transdermal administration, liquid oral solutions, syrups or suspensions in a predetermined unit form, and vials for intravenous or subcutaneous administration.

The pharmaceutical compositions may be formulated in oral forms such as tablets or gelatin capsules, wherein zonisamide; or pramipexole or a pharmaceutically acceptable salt or solvate thereof; or both the active ingredients, are in admixture with a carrier or vehicle. Said carrier or vehicle may include a diluent, such as cellulose, dextrose, lactose, mannitol, sorbitol or sucrose; a lubricant, such as, acid, calcium or magnesium stearate, polyethylene glycol, silica, or talc; and if needed, a binder such as magnesium aluminum silicate, gelatin, methylcellulose, sodium carboxymethylcellulose, or polyvinylpyrrolidone.

Said oral forms may be tablets coated with sucrose or with various polymers.

Alternatively, the tablets can be manufactured by using carriers such as acrylic and methacrylic acid polymers and copolymers; cellulose derivatives such as hydroxypropylethylcellulose; or other appropriate materials. These materials confer a prolonged or delayed activity by progressively releasing a predetermined quantity of zonisamide or pramipexole (or pharmaceutically acceptable salt or solvate thereof).

The oral formulations can also be in form of capsules allowing the extended release of zonisamide; of pramipexole (or pharmaceutically acceptable salt or solvate thereof); or of both the active ingredients.

The pharmaceutical compositions may also be formulated in TTS, such as a patch formulation wherein the active ingredient or the mixture of the active ingredients may comprise adjuvants such as D-sorbitol, gelatin, kaolin, methyl paraben, polysorbate 80, propylene glycol, propyl paraben, povidone, sodium carboxymethylcellulose, sodium polyacrylate, tartaric acid, titanium dioxide, and purified water. A patch formulation may also contain skin permeability enhancer such as lactate esters (e.g., lauryl lactate), triacetin or diethylene glycol monomethyl or monoethyl ether.

In the above pharmaceutical compositions, the preferred (S)-6-propylamino-4,5,6,7-tetrahydro-1,3-benzothiazole-2-amine active ingredient, or a pharmaceutically acceptable salt thereof, is pramipexole base or its dihydrochloride monohydrate.

### EXAMPLES

### Example 1

A Phase I-II clinical study was conducted in parkinsonian subjects receiving oral doses of pramipexole or zonisamide, alone and in combination. The trial was designed as a single-blind study.

The objective of the study was to demonstrate that pramipexole and zonisamide, when administered together at their standard therapeutic doses, can safely normalize concentrations of synuclein species in peripheral blood exosomes.

To be enrolled in the study, male or female participants (40 to 89 years of age) were required to carry the diagnosis of Parkinson's disease or a related synucleinopathic disorder. All subjects signed an informed consent form indicating that they understood the purpose of and procedures required for the study and that they were willing to participate in the study and comply with all study procedures and restrictions. Key criteria for exclusion of a subject from enrollment in the study were as follows:
1. Any clinically relevant acute or chronic disease which could interfere with the subjects' safety during the trial, expose them to undue risk, or interfere with the study objectives.
2. History or presence of gastrointestinal, hepatic, or renal disease or other condition known to interfere with the absorption, distribution, metabolism or excretion of the study medications;
3. History of substance abuse, known drug addiction, or positive test for drugs of abuse or alcohol.
4. History of drug or other significant allergy.
5. Excessive daily consumption of xanthines containing drinks (i.e. >500 mg/day of caffeine).
6. Hospitalization or intake of an investigational drug within 30 days of study entry.

Following baseline clinical and laboratory evaluations, consenting individuals meeting accession criteria were first randomized to either pramipexole or zonisamide treatment. In either case, dosage of the initial drug was gradually increased over the ensuing 6-12 weeks in accordance with current recommendations to each patients' maximum tolerated dose (MTD) or the maximum recommended dose, whichever is lower, and stably maintained for approximately 6 weeks.

Patients then entered the next study phase, lasting about 6-12 weeks when the second study medication was added to their ongoing treatment regimen, in accordance with its recommended titration schedules to their MTD or the maximum recommended dose, whichever is lower. Once safe and tolerable doses of the drug combination was achieved, it was stably maintained for approximately 6 to 12 weeks. Doses of both drugs were then be tapered in accordance with current recommendations and patients were returned to their pre-admission regimen pending discharge from the study.

Drug safety-tolerability was monitored by means of standard clinical and laboratory tests during dose titration, and otherwise at regular intervals throughout the trial. Weekly telephone interviews were generally conducted on those not scheduled for a clinic visit. A final safety check was performed approximately one month after withdrawal of all study medications.

Additionally, venous blood for synuclein and drug assays was collected at baseline and periodically throughout the trial.

Results surprisingly showed that the oral administration of a combination of pramipexole and zonisamide was associated with a tendency to normalize the characteristic alterations in synuclein and synuclein congener concentrations in exosomes collected from peripheral venous blood samples from patients who safely tolerated their therapeutic regimens.

In conclusion, the co-administration of standard approved doses of pramipexole and zonisamide yielded clear peripheral evidence of a drug-combination-induced tendency to normalize synuclein processing indicative of a reduction in toxic species formation in the central nervous system of a type associated with a neuroprotective efficacy that clinically benefits patients suffering from Parkinson's disease or a related synucleinopathy.

These results were totally unexpected since
the ability of zonisamide to augment the biomarker response to pramipexole to a degree that confers clinical patient benefit was never before described, suggested or even anticipated in view of the lack of a rational basis and differences in their pharmacologic properties including mechanism; and
no drug or drug combination, let alone this particular combination, has ever been found to have convincing neuroprotective efficacy in humans with synucleinopathies.

### REFERENCES

Al-Mansoori et al. 2013: Al-Mansoori K M, Hasan M Y, Al-Hayani A, El-Agnaf M, “The role of α-synuclein in neurodegenerative diseases: from molecular pathways in disease to therapeutic approaches”; Curr. Alzheimer Res. 2013 July; 10(6): 559-568.
Arawaka S et al. 2014: Arawaka S, Fukushima S, Sato H, Sasaki A, Koga K, Koyama S, Kato T; “Zonisamide Attenuates a-Synuclein Neurotoxicity by an Aggregation-Independent Mechanism in a Rat Model of Familial Parkinson's Disease”; PLOS One. 2014; 9(2): e89076.
Chen et al. 2016: Chen M, Weiwei Yang W, Li Xin, X, Li Xuran, Wang P, Feng Yue F, Yang H, Chan P, and Yu S; “Age- and brain region-dependent α-synuclein oligomerization is attributed to alterations in intrinsic enzymes regulating α-synuclein phosphorylation in aging monkey brains”; Oncotarget. 2016 Feb. 23; 7(8): 8466-8480.
Corrigan et al. 2000: Corrigan M H, Denahan A Q, Wright C E, Ragual R J, Evans D L; Corrigan M H, Denahan A Q, Wright C E, Ragual R J, Evans D; “Comparison of pramipexole, fluoxetine, and placebo in patients with major depression”; Depress Anxiety. 2000; 11(2):58-65.
Grover et al. 2013: Grover N D, Limaye R P, Gokhale D V, Patil T R; “Zonisamide: a review of the clinical and experimental evidence for its use in Parkinson's disease”; Indian J Pharmacol. 2013 November-December; 45(6):547-55.
Inden et al. 2009: Inden M, Kitamura Y, Tamaki A, Yanagida T, Shibaike T, Yamamoto A, Takata K, Yasui H, Taira T, Ariga H, Taniguchi T; “Neuroprotective effect of the antiparkinsonian drug pramipexole against nigrostriatal dopaminergic degeneration in rotenone-treated mice.”; Neurochem Int. 2009 December; 55(8):760-7.
Jellinger K A 2008a: Jellinger K A, “A critical reappraisal of current staging of Lewy-related pathology in human brain”; Acta Neuropathol. 2008 July; 116(1): 1-16.
Jellinger K A 2008b: Jellinger K A, “Neuropathological aspects of Alzheimer disease, Parkinson disease and frontotemporal dementia”; Neurodegener. Dis. 2008; 5(3-4): 118-121.
Kakimura et al. 2001: Kakimura J, Kitamura Y, Takata K, Kohno Y, Nomura Y, Taniguchi T; “Release and aggregation of cytochrome c and alpha-synuclein are inhibited by the antiparkinsonian drugs, talipexole and pramipexole”; Eur J Pharmacol. 2001 April 6; 417(1-2):59-67.
Kim et al. 2004: Kim S, Seo J H, Suh Y H, “Alpha-synuclein, Parkinson's disease, and Alzheimer's disease”; Parkinsonism Relat. Disord. 2004 May; 10 Suppl. 1: S9-13.
Luo et al. 2016: Luo H T, Zhang J P, Miao F; “Effects of pramipexole treatment on the α-synuclein content in serum exosomes of Parkinson's disease patients”; ExpTher Med. 2016 September; 12(3):1373-1376.
Marques and Outeiro 2012: Marques O, Outeiro T F; “Alpha-synuclein: from secretion to dysfunction and death”; Cell Death Dis. 2012 Jul. 19; 3:e350. doi: 10.1038/cddis.2012.94.
Ono et al. 2013: Ono K, Takasaki J, Takahashi R, Ikeda T, Yamada M; “Effects of antiparkinsonian agents on β-amyloid and α-synuclein oligomer formation in vitro”; JNeurosci Res; 2013 October; 91(10):1371-81).
O'Regan et al 2017: O'Regan G, DeSouza R M, Balestrino R, “Glucocerebrosidase Mutations in Parkinson Disease” J Parkinson's Dis 7 (2017) 411-422-DOI 10.3233/JPD-171092.
Prusiner S B et al. 2015: Prusiner S B, Woerman A L, Mordes D A, Watts J C, Rampersaud R, Berry D B, Patel S, Oehler A, Lowe J K, Kravitz S N, Geschwind D H, Glidden D V, Halliday G M, Middleton L T, Gentleman S M, Grinberg L T, Giles K, “Evidence for α-synuclein prions causing multiple system atrophy in humans with parkinsonism”; ProcNatlAcadSci USA; 2015, Sep. 22; 112(38):E5308-17.
Sano H et al 2015: Sano H, Murata M, Nambu A; “Zonisamide reduces nigrostriatal dopaminergic neurodegeneration in a mouse genetic model of Parkinson's disease”; J Neurochem. 2015 July; 134(2):371-81.
Schneider C S and Mierau J, 1987: Schneider C S, Mierau J “Dopamine autoreceptor agonists: resolution and pharmacological activity of 2,6-diaminotetrahydrobenzothiazole and an aminothiazole analogue of apomorphine”. J. Med Chem. 1987 March; 30(3):494-8.
Shi et al. 2014: Shi M, Liu C, Cook T J, Bullock K M, Zhao Y, Ginghina C, Li Y, Aro P, Dator R, He C, Hipp M J, Zabetian C P, Peskind E R, Hu S C, Quinn J F, Galasko D R, Banks W A, Zhang J; “Plasma exosomal α-synuclein is likely CNS-derived and increased in Parkinson's disease”; ActaNeuropathol. 2014 November; 128(5):639-50. doi: 10.1007/s00401-014-1314-y. Epub 2014 Jul. 6.
Soria et al 2017: Soria F N, Engeln M, Martinez-Vicente M, Glangetas C, Lopez-Gonzales J, Dovero S, Dehay B, Normand E, Vila M, Lopez-Gonzales M J, Favereaux A, Georges F, Lo Bianco C, Bezard E, Fernagut; “Glucocerebrosidase deficiency in dopaminergic neurons induces microglial activation without neurodegeneration”; Hum Mol Genet 2017 July; 26(14):2603-2615.
Stuendl A, Kunadt M, Kruse N, Bartels C, Moebius W, Danzer K M, MollenhauerB, Schneider A; “Induction of alpha-synuclein in aggregate formation by CSF exosomes from patients with Parkinson's disease and dementia with Lewy bodies” Brain 2016, 139; 481-494.
Visanji N P et al. 2016: Visanji N P, Brotchie J M, Kalia L V, Koprich J B, Tandon A, Watts J C, Lang A E; “α-Synuclein-Based Animal Models of Parkinson's Disease: Challenges and Opportunities in a New Era ”; Trends Neurosci. 2016 November; 39(11):750-762.
Yokoyama H et al. 2010: Yokoyama H, Yano R, Kuroiwa H, Tsukada T, Uchida H, Kato H, Kasahara J, Araki T; “Therapeutic effect of a novel anti-parkinsonian agent zonisamide against MPTP (1-methyl-4-phenyl-1,2,3,6-tetrahydropyridine) neurotoxicity in mice”; Metab Brain Dis 2010 June; 25(2):135-43.

1. A method for treating a patient suffering from a synucleinopathy, which comprises treating said patient with zonisamide, in combination with pramipexole or a pharmaceutically acceptable salt or solvate thereof.

2. The method of claim 1 wherein said synucleinopathy is selected from the group consisting of Parkinsons' disease, Parkinson's disease dementia, Lewy body dementia, dementia with Lewy bodies, Alzheimer's disease, the Lewy body variant of Alzheimer's disease, multiple system atrophy, neurodegeneration with brain iron accumulation, and parkinsonian disorders associated with glucocerebrosidase (GBA) mutations.

3. The method of claim 1, wherein said pramipexole or pharmaceutically acceptable salt or solvate thereof is pramipexole dihydrochloride monohydrate.

4. The method of claim 1, wherein zonisamide is administered at a daily dose of from 25 mg to 600 mg, in combination with pramipexole or a pharmaceutically acceptable salt or solvate thereof, at a daily dose 0.375 mg to 20 mg of pramipexole dihydrochloride monohydrate.

5. The method of claim 4, wherein said pramipexole daily dose is equivalent to from 0.375 mg to 6 mg of pramipexole dihydrochloride monohydrate.

6. The method of claim 1, wherein said zonisamide and said pramipexole or pharmaceutically acceptable salt or solvate thereof are each formulated in a pharmaceutical composition in dosage unit form comprising, respectively, said zonisamide and said pramipexole or pharmaceutically acceptable salt or solvate thereof, each in admixture with a pharmaceutical carrier or vehicle.

7. The method of claim 6, wherein said zonisamide is present in said composition in an amount per unit form of from 25 mg to 600 mg.

8. The method of claim 6, wherein said pramipexole or pharmaceutically acceptable salt or solvate thereof is present in said composition in an amount per unit form of from 0.125 mg to 20 mg.

9. The method of claim 8, wherein said pramipexole or pharmaceutically acceptable salt or solvate thereof is present in said composition in an amount per unit form of from 0.125 mg to 6 mg.

10. Zonisamide for use in the treatment of a synucleinopathy in combination with pramipexole or pharmaceutically acceptable salt or solvate thereof.

11. Use of zonisamide for the preparation of a medicament for the treatment of a synucleinopathy in combination with pramipexole or pharmaceutically acceptable salt or solvate thereof.
